(19) United States
(12) Patent Application Publication
(10) Pub. No.: US 2024/0021277 A1
(43) Pub. Date: Jan. 18, 2024

(54) MACHINE LEARNING DRUG EVALUATION USING LIQUID CHROMATOGRAPHIC TESTING

(71) Applicants: William Scott HOPKINS, Waterloo (CA); Centre for Eye and Vision Research Limited, Hong Kong (HK); The Hong Kong Polytechnic University, Hong Kong (HK)

(72) Inventors: Myo Win ZAW, Hong Kong (HK); William Scott HOPKINS, Waterloo (CA); Ming Yan, Allen CHEONG, Hong Kong (HK)

(21) Appl. No.: 17/864,393

(22) Filed: Jul. 14, 2022

(51) Int. Cl. G16C 20/50; G16C 20/70

(52) U.S. Cl. CPC G16C 20/50; G16C 20/70

(57) ABSTRACT

A machine learning system predicts a physicochemical property (e.g., lipophilicity) of candidate small molecules for pharmaceuticals. A machine learning model is constructed that is trained from a database of small molecule physicochemical properties including known lipophilicity and known retention time in a liquid chromatography column to create a learned association between lipophilicity and liquid chromatography retention time. A candidate small molecule having unknown lipophilicity and unknown retention time is applied to a liquid chromatography column. The retention time of the candidate small molecule in the liquid chromatography column is measured. The measured retention time in the liquid chromatography column is applied to the machine learning model to obtain lipophilicity for the candidate small molecule. One or more candidate small molecules having a lipophilicity value from approximately 1 to approximately 3 are selected from the machine learning model. The identified candidate small molecules are tested for pharmaceutical activity.

### FIELD OF INVENTION

The present technology relates to the technical field of computational chemistry and more particularly to machine learning techniques for predicting physicochemical properties of molecules.

### BACKGROUND OF INVENTION

Machine learning has been used to obtain useful insights from large quantities of raw data. Recently, it has been applied to the analysis of chemical compounds, particularly compounds for biomedical applications such as novel drugs. Evaluation of physical chemistry properties has a pivotal role in drug discovery research.

Drug development from a basic idea to the final product is a complex and expensive process. In the early stage, a large number of chemical molecules have to be screened to identify potential compounds that demonstrate some type of chemical activity. However, it is not feasible to search these potential compounds for drug candidates by traditional methods. The cost of bringing a single drug from the initial screening to a clinical trial averages hundreds of millions of dollars over many years; Therefore, improved techniques for identifying strong candidate drug molecules are needed. The present invention addresses this need.

### SUMMARY OF THE INVENTION

Lipophilicity, logP, the octanol-water partition coefficient, is one of the indicators in assessing the use of a target molecule as a drug since it indicates absorption, distribution, metabolism, excretion, toxicity, and potency of the target molecule as a drug. The present invention is directed to accurately predicting lipophilicity and other physicochemical properties of molecules in order to identify candidate drugs for further testing.

In one aspect, the present invention provides a machine learning system for predicting the lipophilicity of candidate small molecules for pharmaceuticals. A machine learning model is constructed that is trained from a database of small molecule physicochemical properties including known lipophilicity and known retention time in a liquid chromatography column to create a learned association between lipophilicity and liquid chromatography retention time.

A candidate small molecule having an unknown lipophilicity and unknown retention time is applied to a liquid chromatography column. The retention time of the candidate small molecule in the liquid chromatography column is measured.

The measured retention time in the liquid chromatography column is applied to the machine learning model to obtain a lipophilicity for the candidate small molecule. One or more candidate small molecules having a lipophilicity value from approximately 1 to approximately 3 is selected from the machine learning model. The identified candidate small molecules are tested for pharmaceutical activity.

In another aspect, the machine learning system uses a database of small molecule physicochemical properties including the acid dissociation constant (pKa), cell permeability, and polar surface area.

In another aspect, the machine learning model comprises a Random Forest Regression algorithm.

In another aspect, the machine learning model comprises a Gradient Boosting algorithm.

In another aspect, the machine learning model comprises a Support Vector Machine algorithm.

In another aspect, herein the machine learning model comprises a Deep Neural Network algorithm.

In another aspect, the machine learning model is further trained by one or more indicators of computed molecular descriptors for the candidate small molecule.

In another aspect, the indicators of computed molecular descriptors include one or more computed parameters of mass, dipole moment, atomic composition, Morgan fingerprint, Tanimoto similarity.

In another aspect, the machine learning model is further trained from one or more of an indicator of mass spectrometry or ion mobility.

In another aspect, the indicator of mass spectrometry or ion mobility is a mass-to-charge ratio (m/z) or collision cross-section (CCS).

In another aspect, the machine learning model is further trained using an indicator of a liquid chromatography solvent system or column stationary phase material.

### BRIEF DESCRIPTION OF THE DRAWINGS

FIG. 1 depicts the P-Chem property prediction flow chart. Dashed arrows depict the transformation and extraction of the information from the public database. Solid arrows represent the learning process.

FIGS. 2a-2f shows the distribution of physicochemical properties of the extracted data and experimental retention time from SMRT data set for (FIG. 2a) LogD, (FIG. 2b) LogP, (FIG. 2c) PSA, (FIG. 2d) a_pKa, (FIG. 2e) b_pKa, and (FIG. 2f) Exp_RT.

FIG. 3 depicts the heat map of Pearson's correlation between experimental retention time and physicochemical properties. The scale on the right indicates the correlation length.

FIG. 4 is the correlation matrix plot with significance levels between experimental retention time and physicochemical properties.

FIGS. 5a-5h show the learning curves for four models on the left column and Experimental vs predicted logP on the left column for MLP in (FIG. 5a) and (FIG. 5b), for SVM in (FIG. 5c) and (FIG. 5d) for GB in (FIG. 5e) and (FIG. 5f), and for RF in (FIG. 5g) and (FIG. 5h).

FIGS. 6a-6d shows the complete set of performance metrics on the test set using different machine learning models. (FIG. 6a) MSE, (FIG. 6b) RMSE, (FIG. 6c) MAE and (FIG. 6d) R2. The hatched and solid bar represents the model without RT and with RT.

FIG. 7 is a SHAP summary plot showing the impact of descriptors (the top 20) on the model. One dot represents one molecule, and the dots stack up to show its density.

### DETAILED DESCRIPTION

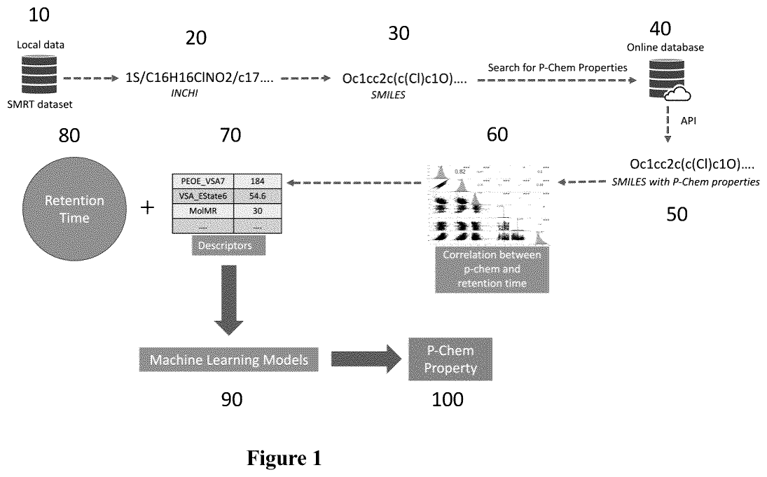

Turning to the drawings in detail, FIG. 1 depicts an overview of the physicochemical property prediction machine learning system of the present invention. Initially, a machine learning model is trained to learn the correlation between physicochemical properties such as lipophilicity (logP), acid dissociation constant (pKa) and polar surface area and a liquid chromatography measurable property such as liquid chromatography retention time. In the example used to describe the invention, lipophilicity (logP) is the property that is correlated to the liquid chromatography retention time. However, it is understood that the correlation between other physicochemical properties of target molecules and liquid chromatography measurable properties can be used in the training and the trained model used to predict another physicochemical property of the target molecule.

In the training, various public domain databases are use that have both the known values of the selected physicochemical properties and known liquid chromatography measurable properties such as liquid chromatography retention time. Once a particular trained machine learning algorithm has been trained to learn the correlation between various the desired physicochemical property and liquid chromatography retention time, the trained machine learning algorithm is able to predict a physicochemical property for an unknown compound based on a measured liquid chromatography retention time for that unknown compound.

At position 10, in FIG. 1, a data set of target small molecules is procured. As seen in FIG. 1, position 10, a SMRT (small molecule retention time) data set is used. At position 20 in FIG. 1, the initial and final molecule's International Chemical Identifier (InChI) is applied to the data set to remove inconsistent data, such as molecules having multiple different structures for the same molecule.

In order to enter small molecule data in a standard data format used in cheminformatics and molecular data sets, the Simplified Molecular Input Line Entry System (SMILES) is employed at position 30 in FIG. 1. SMILES encodes atoms and their molecular bonds using a text notation with a grammar that ensures a proper interpretation of the data by a machine learning model. In SMILES, a single line of text with a fixed character length denotes the atoms and their bonds. A RDKit package was used to read the SMILES data for later use in training the machine learning models.

Once the SMILES data has been read, the RDKit package searches publicly available online databases (e.g., open-source databases such as ChEMBL, a manually curated chemical database of bioactive molecules with drug-like properties) for physicochemical properties such as logP/logD, a_pKa, b_pKa, and PSA at position 40 in FIG. 1. According to statistical analysis performed in the present invention, it has been determined that there is a moderate correlation between the liquid chromatographic retention time and scraped physicochemical properties. These physicochemical properties, along with known liquid chromatographic retention times, are added to the SMILES data at position 50 in FIG. 1.

At position 60, a selected machine learning model (to be discussed in further detail, below) is trained with the SMILES data having the added physicochemical properties. In this manner, the selected machine learning model correlates a selected physicochemical property, such as lipophilicity, with the liquid chromatographic retention time.

At position 80, a target molecule is tested in a liquid chromatography column; the retention time of the target molecule in the liquid chromatography column is measured. The measured retention time is added at position 70 to known physicochemical properties of the target molecule that is measured at position 80.

These properties and the measured property are sent to the trained machine learning model at position 90. Using the learned correlation between a desired physicochemical property to be determined (such as lipophilicity) and liquid chromatographic retention time, the model uses the measured liquid chromatographic retention time to predict the desired physicochemical property (e.g., lipophilicity).

When using the machine learning system to predict lipophilicity, target molecules having a predicted lipophilicity ranging from approximately 1 to approximately 3 are selected as candidates for pharmaceutical use. These values correlate to a desirable lipophilicity where the lipophilicity is sufficiently low for the molecule to enter the bloodstream and sufficiently high for the molecule to passively cross cell membranes.

The prediction models are developed by using one or more machine learning algorithms. A variety of machine learning algorithms may be selected. In one embodiment, a support vector machine (SVM) SVMs use sets of supervised learning methods for classification, regression, and outliers detection of large data sets. SVMs typically find a separator between different categories of data (a “hyperplane”) such that the data can be partitioned into classes. In this manner, a new data point is placed into a correct category for predictive analysis.

A multilayer perceptron (MLP) technique may also be used. MLP is a feedforward neural network that uses a set of inputs to generate a set of outputs having input nodes connected as a directed graph between the input and output layers. In training, back-propagation is used to create a prediction approach based on decision trees.

Gradient boosting (GB) is a machine learning technique used for data classification. When trained, a group of prediction models, such as decision trees, are provided for predicting the target molecule's physicochemical properties based on its liquid chromatographic retention time.

A random forest (RF) machine learning model may also be used. It constructs decision trees or forests during training. Data input to the trained random forest model is classified as the class selected by the most decision trees. K-nearest neighbors (k-NN) is a further machine learning model that can perform the physicochemical property prediction of the present invention. K-NN uses proximity to make predict the classification of data.

In the present invention, molecular fixed representations as described above are used to train one or more of the above models. Then, the models are extensively tested and compared to determine the most accurate model in terms of predictive ability. It is noted that a model trained on the dataset which is appended with a retention time as a descriptor typically shows better performance than those trained on a dataset without retention time. The training of the machine learning module may be improved if the training data set includes structure patterns that have a high frequency, reducing the number of outliers. In this way, the model can reinforce the correlation among physicochemical properties such as lipophilicity, and retention time.

### EXAMPLES

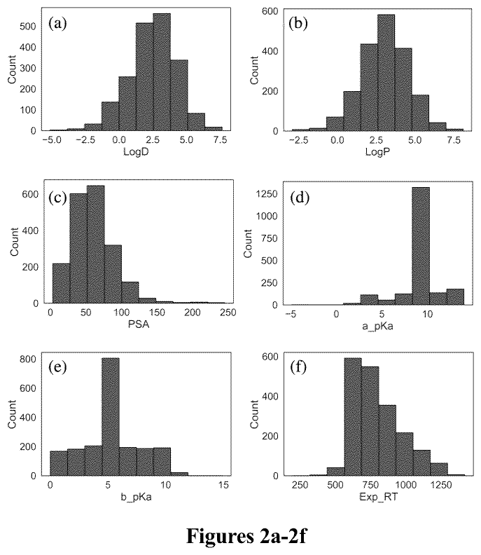

As shown in FIG. 1, the training dataset is prepared in two stages. In stage one, a search is conducted in ChEMBL for the molecule in the SMRT dataset after converting from INCHI to SMILE code. In stage two, identification of physicochemical properties that are correlated with retention time is conducted. In order to see the distribution of each scraped property, the histograms are provided in FIG. 2.

### Molecular Representation

The extracted compounds were used to calculate molecular descriptors using the free and open-source software RDKit (Version: 2021.03.4). The MolFromSmiles method from RDKit was applied to convert from SMILES to molecular objects. A total of 205 molecular descriptors were calculated, including atom-type E-state indices, molecular weight, number of valence electrons, fragmental, number of rotatable bonds, number of ring counts, and other physicochemical descriptors. Prior to the development of the machine learning models, all the features are pretreated as follows: (1) the features with low variance (<0.05), missing values, and zeros values were removed (2) the features correlated with another feature (<0.95) were removed (3) the retained features are scaled to mean values of 0 and variance of 1.

### Machine Learning Algorithms

Four ML algorithms (SVM, MLP, GB, RF) were used to develop the descriptor-based models. These ML algorithms were implemented in the scikit-learn package (Version: 0.24.2) of Python (Version: 3.9.6×64). For finding the ideal hyper-parameters, the hyperopt package (Version: 0.2.5) has been opted. Jupyter Notebook, visual studio code, and Ubuntu Linus systems have been used for running the machine learning models. Details of the results for each machine learning model are discussed below.

### Performance Evaluation Metrics

The following series of metrics compare the performance of models on the test data set.

### Mean Square Error (MSE)

Mean square error is the average of the squared of the difference between the real and predicted values. The lower the value of MSE, the better the performance of the model.

$$M S E = \frac{1}{n} {Σ}_{i = 1}^{n} {( {y}_{i} - {\hat{y}}_{i} )}^{2}$$

Where yᵢ and ŷᵢ are real and predicted values, n is the number of the sample point.

### Root Mean Square Error (RMSE)

Root mean square error is the square root of the average of the squared error which is between the real and predicted values.

$$R M S E = \sqrt{\frac{1}{n} {Σ}_{i = 1}^{n} {( {y}_{i} - {\hat{y}}_{i} )}^{2}}$$

### Mean Absolute Error (MAE)

Mean absolute error is the average of the absolute difference between real and predicted values.

$$M A E = \frac{1}{n} {Σ}_{i = 1}^{n} \vert {y}_{i} - {\hat{y}}_{i} \vert$$

### Coefficient of Determination (R²)

The coefficient of determination indicates whether the model is a good fit.

$${R}^{2} = 1 - \frac{{Σ ( {y}_{i} - {\hat{y}}_{i} )}^{2}}{{Σ ( {y}_{i} - {\hat{y}}_{i} )}^{2}}$$

### Dataset Splitting

For dataset splitting and model training, the extracted dataset was randomly divided into training 80%, validation 10%, and testing 10%. The validation set was used for optimizing the hyper-parameters. The hyperopt optimization technique was used for finding the best combination of hyper-parameters. It uses a form of Bayesian optimization to identify the best parameter for a given model. Then, 50 independent runs with different random seed for data splitting (8:1:1) were performed to reduce the randomness of data splitting and the average result of all the run was reported. Since all the models are for regression tasks, they are evaluated mainly by root mean squared error (RMSE).

### Distribution of Extracted Properties and RT

The P-chem properties (logP, logD, PSA, a_pKa, b_pKa) were explored to find a good correlation and RT from the SMRT dataset which would thus enable LCMS-based properties screening for drug discovery. First for all, the targeted P-Chem properties were extracted from ChEMBL database. One extract from ChEMBL produced 2070 hits (for 10000 compounds) with the above-mentioned P-Chem properties.

As depicted in FIG. 2, the logP and logD are approximately normally distributed, where logP spans from −2.91 to 8.1 with the median of 3.05 while logD ranges from −5.12 to 7.63 with a median of 2.58. However, the data for PSA is moderately positively skewed (skewness˜1.20) since the mean value is greater than the median. For a_pKa and b_pKa, each has one peak around 10 and 5. For retention time, it is normally distributed with values from 196.8 to 1418.10 seconds.

### Statistical Analysis

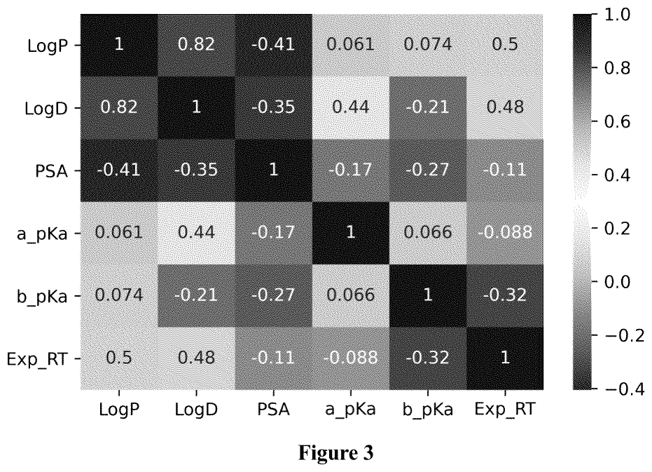

To evaluate the correlation between the RT and P-Chem, Pearson correlation was used. As shown as in FIG. 3, LogP and LogD are positively correlated with RT. However, the correlation of PSA, a_pKa and b_pKa was significantly negative.

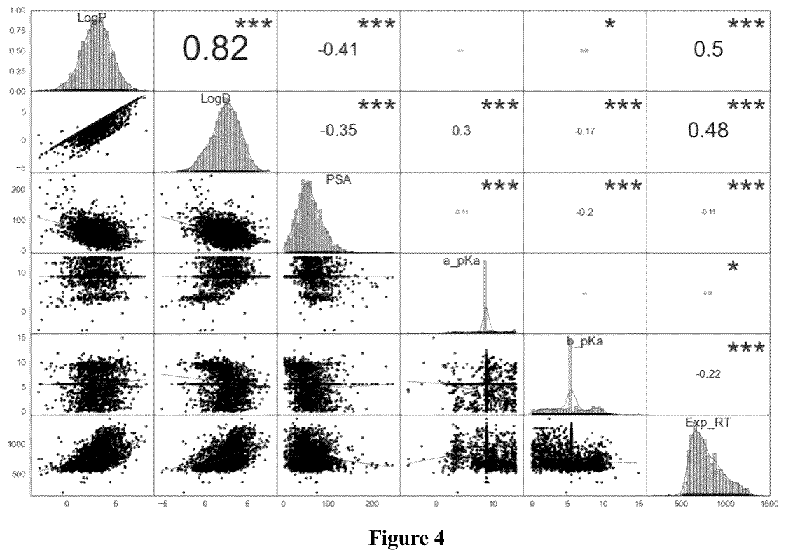

Because of the moderate correlation between RT and LogP and LogD data, the effect of adding RT as additional descriptor to the models for predicting the logP values was determined. The P-Chem properties were further analyzed in order to confirm the correlation. The correlation matrix plot with significance level confirms that there is a moderate correlation between RT and logP and logD in FIG. 4.

There are two types of molecular representations which can be used as input data to build predictive models for molecular properties: fixed representations and learned representations. Fixed representations such as fingerprints and descriptors have been widely used. In the present study, RDKit python library was used to generate 2-D molecular descriptors. The optimized hyper-parameters are used for depicting the learning curve to understand the models' generalization ability.

### Result of SVM

Support vector machine (SVM) is a machine learning model based on statistical learning theory. In this embodiment, the linear kernel was used. For this kernel in SVM, one hyper-parameter is needed for optimization: regularization parameter (C). The regularization parameter C from 0.1 to 100 was optimized.

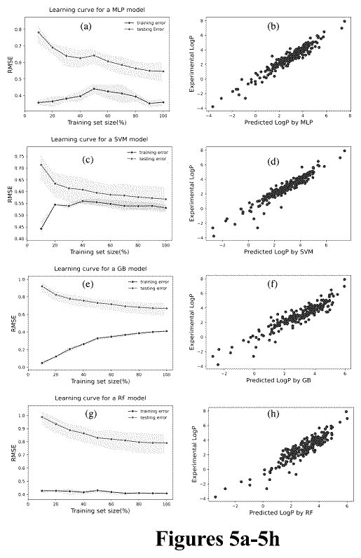

The following RMSE values for SVM are obtained after performing the optimization. The RMSE value for the SVM model without RT is 0.513 and 0.500 for the model with RT. It can be seen that the RMSE is reduced by more training data in the learning curve in FIG. 5(c). The learning curve for SVM was close to converged at maximum training set size suggest that it could not be improved any further by adding more samples.

### Result of MLP

Multilayer perceptron (MLP) is a fully connected artificial neural network (ANN) trained using backpropagation. It uses three layers of nodes: (1) input layer, (2) hidden layers, and (3) output layer. Each neuron in MLP uses a nonlinear activation function except the input layer. It mimics the behavior of biological neurons in the brain. In this embodiment, the following hyper-parameters were optimized: hidden layer size ((150,100,50), (120,80,40), (100,50,30)), max_iter ([5, 10 ,50 ,100, 200]), activation ‘relu’, ‘tanh’, ‘logistic’), solver (‘sgd’, ‘adam’), alpha (0.0001, 0.05), learning rate (‘constant’, ‘adaptive’). The other important hyper-parameters were fixed.

In the best MLP configuration with 3 dense layers having 150, 100, 50 neurons respectively, an RMSE of 0.502 without RT and an RMSE of 0.494 with RT were achieved. The MLP is slightly better than SVM. From the learning curve for the MLP model in FIG. 5(a), the gap between training error and testing error tends to reduce for larger training set size. This means that the MLP model shows a good learning capacity. Moreover, a good fit was found between the real and predicted results.

### Result of GB

Gradient boosting (GB) is one of the powerful learning algorithms in building the predictive model. There are two types of errors in machine learning models: bias error and variance error. This tree-based ensemble method produces high prediction accuracy by minimizing the previous model's bias error. In the training of GB, the following hyper-parameters were optimized: learning rate (0.01 to 0.2), n_estimators (50,100,200,300,400,500), subsample (0.7 to 1.0), min_samples_split (0.1 to 1.0) and min_samples_leaf (0.1 to 0.5).

This ensemble model produces an RMSE of 0.622 without RT and 0.610 with RT which is worse than SVM and MLP. The learning curve for GB in FIG. 5(e) indicated that the GB model has the potential to be converged. Besides, the difference between training and testing narrows at larger data size suggesting that the GB model improves the generalization.

### Result of RF

Random forest is another tree-based learning model with an ensemble learning method for classification and regression. The random forest establishes the outcome based on the prediction of the decision trees. The prediction is done by returning the mean or average of the output of the various trees. In the training of RF, the following hyper-parameters were optimized: n_estimators (50,100,200,300,400), max_depth (3 to 12), min_samples_leaf (1, 3, 5, 10, 20, 50), min impurity decrease (0 to 0.01) and max_features (‘sqrt’, ‘log2’, 0.7, 0.8, 0.9).

The performance of RF model is the worst among the models with an RMSE value of 0.792 without RT and 0.744 with RT. From the learning curve for RF in FIG. 5(e), it is worth noting that the bias was progressively reduced during the learning process. Due to the complexity of the random forest model, the model was far from convergence at maximum sample size thus the model cannot be improved by adding more training data. Thus, RF has the worst performance of all the models.

### Comparison

Table 1 presents the performance results for the four tested regression models: SVM, random forest (RF), gradient boosting, and multi-layer perceptron (MLP). MSE, RMSE, MAE, R2 values were used to evaluate the performance of regression models. To evaluate the models in a reliable way, 50 independent runs with different random seeds for train, validation, and test splitting at the ratio of 8:1:1 were conducted. As shown in Table 2, it can be recognized that SVM and MLP models give slightly better performance than the other models in terms of RMSE values which is in good agreement with the previous single random split in Table 1.

TABLE 1
Performance comparison (MSE, RMSE, MAE, R²) of the models (single
random run) without retention time and with retention time:

| | Metric | Model | Training | Validation | Test |
|---|---|---|---|---|---|
| No RT | MSE | SVM | 0.235 | 0.253 | 0.293 |
| | | MLP | 0.123 | 0.193 | 0.252 |
| | | GB | 0.143 | 0.371 | 0.434 |
| | | RF | 0.218 | 0.659 | 0.666 |
| RT | MSE | SVM | 0.228 | 0.237 | 0.275 |
| | | MLP | 0.105 | 0.221 | 0.222 |
| | | GB | 0.142 | 0.326 | 0.332 |
| | | RF | 0.157 | 0.727 | 0.481 |
| No RT | RMSE | SVM | 0.485 | 0.503 | 0.541 |
| | | MLP | 0.352 | 0.439 | 0.502 |
| | | GB | 0.378 | 0.609 | 0.659 |
| | | RF | 0.467 | 0.812 | 0.816 |
| RT | RMSE | SVM | 0.477 | 0.487 | 0.525 |
| | | MLP | 0.324 | 0.471 | 0.472 |
| | | GB | 0.377 | 0.571 | 0.576 |
| | | RF | 0.396 | 0.852 | 0.694 |
| No RT | MAE | SVM | 0.328 | 0.336 | 0.338 |
| | | MLP | 0.263 | 0.340 | 0.364 |
| | | GB | 0.274 | 0.426 | 0.423 |
| | | RF | 0.337 | 0.622 | 0.599 |
| RT | MAE | SVM | 0.317 | 0.335 | 0.375 |
| | | MLP | 0.244 | 0.343 | 0.353 |
| | | GB | 0.263 | 0.429 | 0.441 |
| | | RF | 0.287 | 0.600 | 0.524 |
| No RT | R² | SVM | 0.897 | 0.906 | 0.869 |
| | | MLP | 0.947 | 0.909 | 0.888 |
| | | GB | 0.939 | 0.837 | 0.801 |
| | | RF | 0.906 | 0.737 | 0.689 |
| RT | R² | SVM | 0.902 | 0.902 | 0.881 |
| | | MLP | 0.954 | 0.899 | 0.912 |
| | | GB | 0.939 | 0.866 | 0.849 |
| | | RF | 0.932 | 0.749 | 0.762 |

Among all the models, MLP gives the best performance of RMSE 0.494 to test sets. SVM is slightly worse than MLP with RMSE 0.500. GB and RF offer worse predictions than SVM and MLP with RMSE 0.610 and RMSE 0.744. In terms of performance efficiency, SVM and MLP only need a few seconds to train a model. Hence, this implies that the MLP and SVM method predicts the data very efficiently.

In previous studies, an MLP model using the DeepChem database demonstrated the best performance of RMSE=0.627±0.02. LogP was predicted with RMSE=0.61 with a set of 11 drug-like molecules provided by SAMPL6. The present invention was able to build effective regressors that had a better performance than previously published studies. It is understood that the performance of any model depends on the number, diversity, and data sizes. The present invention demonstrates that adding descriptors can improve the performance of a machine learning model; in the above example, the experimental retention time was added as a descriptor to the training set in order to see the effect of RT. The effect of adding RT was summarized in Table 2, below.

TABLE 2
Performance comparison (MSE, RMSE, MAE, R²)
of the models (50 times independent runs) without
retention time and with retention time:

| | Metric | Model | Training | Validation | Test |
|---|---|---|---|---|---|
| No | MSE | SVM | 0.240 ± 0.009 | 0.268 ± 0.064 | 0.266 ± 0.061 |
| RT | | MLP | 0.133 ± 0.018 | 0.256 ± 0.055 | 0.255 ± 0.058 |
| | | GB | 0.149 ± 0.006 | 0.413 ± 0.071 | 0.389 ± 0.056 |
| | | RF | 0.215 ± 0.007 | 0.654 ± 0.100 | 0.631 ± 0.080 |
| RT | MSE | SVM | 0.227 ± 0.009 | 0.256 ± 0.058 | 0.253 ± 0.057 |
| | | MLP | 0.115 ± 0.020 | 0.247 ± 0.045 | 0.247 ± 0.059 |
| | | GB | 0.138 ± 0.005 | 0.391 ± 0.066 | 0.375 ± 0.057 |
| | | RF | 0.156 ± 0.005 | 0.578 ± 0.08 | 0.557 ± 0.074 |
| No | RMSE | SVM | 0.490 ± 0.009 | 0.514 ± 0.059 | 0.513 ± 0.058 |
| RT | | MLP | 0.364 ± 0.024 | 0.504 ± 0.053 | 0.502 ± 0.056 |
| | | GB | 0.386 ± 0.007 | 0.640 ± 0.055 | 0.622 ± 0.045 |
| | | RF | 0.464 ± 0.007 | 0.806 ± 0.062 | 0.792 ± 0.051 |
| RT | RMSE | SVM | 0.477 ± 0.009 | 0.503 ± 0.055 | 0.500 ± 0.056 |
| | | MLP | 0.338 ± 0.028 | 0.495 ± 0.045 | 0.494 ± 0.056 |
| | | GB | 0.371 ± 0.007 | 0.623 ± 0.053 | 0.610 ± 0.047 |
| | | RF | 0.395 ± 0.006 | 0.758 ± 0.056 | 0.744 ± 0.050 |
| No | MAE | SVM | 0.325 ± 0.003 | 0.354 ± 0.024 | 0.352 ± 0.024 |
| RT | | MLP | 0.272 ± 0.016 | 0.363 ± 0.027 | 0.362 ± 0.025 |
| | | GB | 0.277 ± 0.004 | 0.448 ± 0.027 | 0.443 ± 0.024 |
| | | RF | 0.338 ± 0.004 | 0.594 ± 0.038 | 0.590 ± 0.033 |
| RT | MAE | SVM | 0.321 ± 0.003 | 0.349 ± 0.024 | 0.348 ± 0.022 |
| | | MLP | 0.256 ± 0.021 | 0.358 ± 0.026 | 0.356 ± 0.029 |
| | | GB | 0.262 ± 0.004 | 0.440 ± 0.026 | 0.435 ± 0.025 |
| | | RF | 0.286 ± 0.004 | 0.558 ± 0.034 | 0.555 ± 0.031 |
| No | R² | SVM | 0.897 ± 0.003 | 0.883 ± 0.024 | 0.882 ± 0.031 |
| RT | | MLP | 0.943 ± 0.007 | 0.888 ± 0.021 | 0.886 ± 0.030 |
| | | GB | 0.936 ± 0.002 | 0.819 ± 0.028 | 0.828 ± 0.029 |
| | | RF | 0.908 ± 0.002 | 0.715 ± 0.031 | 0.722 ± 0.038 |
| RT | R² | SVM | 0.903 ± 0.003 | 0.888 ± 0.023 | 0.887 ± 0.029 |
| | | MLP | 0.950 ± 0.008 | 0.892 ± 0.017 | 0.890 ± 0.030 |
| | | GB | 0.941 ± 0.002 | 0.829 ± 0.025 | 0.834 ± 0.031 |
| | | RF | 0.933 ± 0.002 | 0.748 ± 0.027 | 0.754 ± 0.036 |

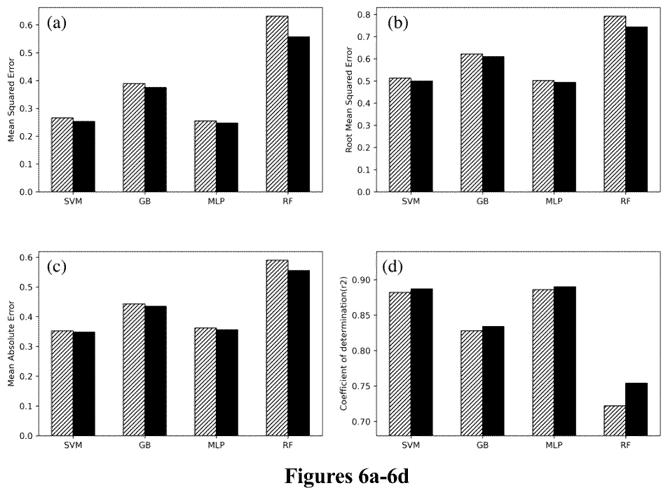

As can be seen from Table 2, all the models with RT perform better than the model without RT. The MLP model with RT performed better than the MLP model without RT in terms of MSE improvement of˜0.010(From 0.255 to 0.247), RMSE improvement of ˜0.010(0.502 to 0.494), MAE improvement of˜0.040(from 0.362 to 0.356), R2 improvement of˜0.010(from 0.886 to 0.890). For SVM, the improvement is MSE value of˜0.013(from 0.266 to 0.253), RMSE value˜0.013(from 0.513 to 0.500), MAE of ˜0.004(from 0.352 to 0.348), and R2 value of˜0.001(from 0.882 to 0.887). Without RT, the SVM model offers comparable performances with MLP. The same trend can be found in the rest of the model: the RMSE improvements were detected for GB and RF models when RT was added as a descriptor. FIG. 5 shows all the learning curves and their respective predictions. To confirm the detail of prediction, the histogram of prediction errors and R2 score are plotted in FIG. 6. The complete set of metrics score on the test set using different machine learning models without RT and with RT indicated the models with RT are superior to the models without RT. Thus, the additional descriptor did have a positive impact on the test set performance.

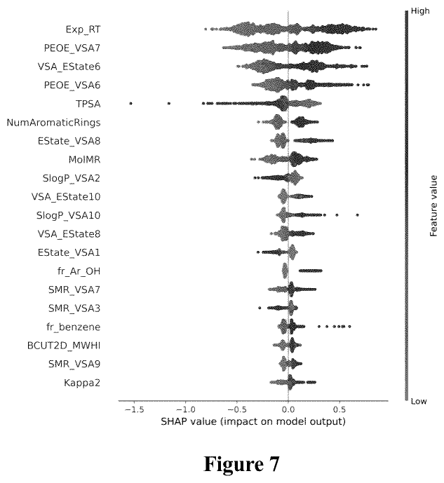

The descriptors were further analyzed to determine the greatest contribution to the models by using the SHAP (Shapley Additive exPlanations) method. The GB model was used as an example. FIG. 7 highlights the top 20 representative molecular descriptors which contribute most to the models. The dots represent the number of molecules in the data set. The feature values and SHAP value from FIG. 7 illustrate that the values of RT have the greatest influence on predicted values of logP.

Predicting logP plays an important role in assessing the molecule for a drug candidate. As set forth in the Example above adding RT has been demonstrated to improve the predictive performance of the machine learning in terms of accuracy and computability.

The present invention can be embodied in a system, a method, and/or a computer program product. The computer program product can include a computer readable storage medium that carries a computer readable program instruction for implementing the various aspects of the present invention to the processor.

The computer readable storage medium can be a tangible device that can hold and store instructions used by the instruction execution device. Computer readable storage media can be, for example, but is not limited to, an electrical storage device, a magnetic storage device, a light storage device, an electromagnetic storage device, a semiconductor storage device, or an arbitrary combination of the above. More specific examples of computer readable storage media (non-exhaustive lists) include: Portable Computer Disc, Hard Disk, Random Access Memory (RAM), read-only memory (ROM), removable programmable read-only memory (EPROM Or flash memory), static random access memory (SRAM), portable compression disk read only memory (CD-ROM), digital multi-function disk (DVD), memory stick, floppy disk, mechanical encoding device, and any suitable combination of the above. The computer readable storage medium used herein is not interpreted as an instantaneous signal itself, such as radio waves or other free propagation electromagnetic waves, electromagnetic waves propagated by waveguide or other transport medium (e.g., through the optical pulse of the fiber optic cable).

The computer program instruction used to perform the operation of the present invention may be a compilation instruction, an instruction set architecture (ISA) instruction, machine instruction, machine-related instruction, microcode, firmware instruction, status setting data, or in one or more programming languages. Any combination of source code or target code, the programming language, may be used, including object-oriented programming languages, such as SmallTalk, C++, Python, etc., and conventional process programming languages such as “C” languages or similar programming languages. Computer readable program instructions can be performed on the user's computer, partially executed on the user's computer, execute as a separate package, partially performed on the remote computer on the remote computer, or on the remote computer or server implement. In the case involving remote computers, remote computers can connect to user computers by any kind of network, including a local area network (LAN) or WAN (WAN), or can be connected to external computers (e.g., using Internet service providers through the Internet via the Internet connect). In some embodiments, personalized electronic circuitry, such as a programmable logic circuit, field programmable gate array (FPGA), or programmable logic array (PLA), by using a state information of the computer readable program instruction may be used.

Embodiments of the present invention have been described above, and the above description is exemplary, non-exhaustive, and is not limited to the disclosed embodiments. Many modifications and changes in the art will be apparent to those of ordinary skill in the art without departing from the scope and spirit of the various embodiments described. The selection of the terms used herein is intended to be the best explanation of the principles, practical applications of the various embodiments, or techniques for techniques in the market, or other one of ordinary skill in the art will appreciate the embodiments disclosed herein. The scope of the invention is defined by the appended claims.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or ±0.5% of the given value or range. The range may be indicated herein as from one endpoint to another endpoint or between two endpoints. Unless otherwise specified, all the ranges disclosed in the present disclosure include endpoints. The term “substantially coplanar” may refer to two surfaces within a few micrometers (um) positioned along the same plane, for example, within 10 μm, within 5 μm, within 1 μm, or within 0.5 μm located along the same plane. When reference is made to “substantially” the same numerical value or characteristic, the term may refer to a value within ±10%, ±5%, ±1%, or ±0.5% of the average of the values.

1. A machine learning system for predicting a physicochemical property of candidate small molecules for pharmaceuticals comprising:
constructing a machine learning model trained from a database of small molecule physicochemical properties including a known physicochemical property for each molecule and a known retention time in a liquid chromatography column to create a learned association between the physicochemical property and liquid chromatography retention time.
applying a candidate small molecule having an unknown physicochemical property and unknown retention time to a liquid chromatography column and measuring the retention time of the candidate small molecule in the liquid chromatography column.
applying the measured retention time in the liquid chromatography column to the machine learning model to obtain a predicted physicochemical property for the candidate small molecule.
selecting one or more candidate small molecules having a target value of the physicochemical property from the machine learning model;
testing the selected candidate small molecules for pharmaceutical activity.

2. The machine learning system of claim 1, wherein the database of small molecule physicochemical properties is a small molecule retention time (SMRT) dataset including International Chemical Identifier (InChi) codes, and extracted data are converted to Simplified Molecular Input Line Entry System (SMILES) notation to extract physico-chemical properties as a query to a ChEMBL database.

3. The machine learning system of claim 1, wherein the physicochemical property is lipophilicity.

4. The machine learning system of claim 3, wherein the target lipophilicity is between approximately 1 and approximately 3.

5. The machine learning system of claim 1, where the database of small molecule physicochemical properties includes acid dissociation constant (pKa) and polar surface area.

6. The machine learning system of claim 1, wherein the machine learning model comprises a Random Forest Regression algorithm.

7. The machine learning system of claim 1, wherein the machine learning model comprises a Gradient Boosting algorithm.

8. The machine learning system of claim 1, wherein the machine learning model comprises a Support Vector Machine algorithm.

9. The machine learning system of claim 1, wherein the machine learning model comprises a Deep Neural Network algorithm.

10. The machine learning system of claim 1, wherein the machine learning model is further trained by one or more indicators of computed molecular descriptors for the candidate small molecule.

11. The machine learning system of claim 10, wherein the indicators of computed molecular descriptors include one or more computed parameters of mass, dipole moment, atomic composition, Morgan fingerprint, Tanimoto similarity.

12. The machine learning system of claim 1, wherein the machine learning models are trained without the experimentally measured retention time descriptor in the liquid chromatography column to predict the lipophilicity.

13. The machine learning system of claim 1, wherein the machine learning models are trained with the experimentally measured retention time descriptor in the liquid chromatography column to predict the lipophilicity.
